SEQUENCE LISTING (18 sequences)

SEQ ID NO: 1 - DNA, 22 bp, Artificial Sequence
  as primer for PCR
catgttttctgacggcaacttc

SEQ ID NO: 2 - DNA, 20 bp, Artificial Sequence
  as primer for PCR
agggccttgagcaccagttt

SEQ ID NO: 3 - DNA, 21 bp, Artificial Sequence
  as primer for PCR
tgtcgcccttttctactttgc

SEQ ID NO: 4 - DNA, 20 bp, Artificial Sequence
  as primer for PCR
tgggatgcctttgtggaact

SEQ ID NO: 5 - DNA, 23 bp, Artificial Sequence
  as primer for PCR
gagacagccaggagaaatcaaac

SEQ ID NO: 6 - DNA, 22 bp, Artificial Sequence
  as primer for PCR
tatggccccagcatgcgacctc

SEQ ID NO: 7 - DNA, 20 bp, Artificial Sequence
  as primer for PCR
taccagtggaggccgacttc

SEQ ID NO: 8 - DNA, 29 bp, Artificial Sequence
  as primer for PCR
tccacagcacctggttattattcttggcg

SEQ ID NO: 9 - DNA, 21 bp, Artificial Sequence
  as primer for PCR
cctgcaccaccaactgcttag

SEQ ID NO: 10 - DNA, 21 bp, Artificial Sequence
  as primer for PCR
cagtcttctgggtggcagtga

SEQ ID NO: 11 - DNA, 25 bp, Artificial Sequence
  as primer for PCR
catccatgacaactttggtatcgtg

SEQ ID NO: 12 - DNA, 22 bp, Artificial Sequence
  as primer for PCR
agtgcttttgttgtggtggaaa

SEQ ID NO: 13 - DNA, 20 bp, Artificial Sequence
  as primer for PCR
ggcccaaaacaaagaagcaa

SEQ ID NO: 14 - DNA, 27 bp, Artificial Sequence
  as primer for PCR
cctggtcagaacacaggcgacactttc

SEQ ID NO: 15 - DNA, 21 bp, Artificial Sequence
  as primer for PCR
aacaggcaagcagcaaagttg

SEQ ID NO: 16 - DNA, 20 bp, Artificial Sequence
  as primer for PCR
gggtgtttccggtctgagaa

SEQ ID NO: 17 - DNA, 27 bp, Artificial Sequence
  as primer for PCR
ccccagtggtgctcagaccagagattc

SEQ ID NO: 18 - DNA, 20 bp, Artificial Sequence
  as primer for PCR
caaagcgactggatgaacca

(19) United States
(12) Patent Application Publication
(10) Pub. No.: US 2016/0089410 A1
(43) Pub. Date: Mar. 31, 2016

(54) COMPOSITION FOR NOURISHING INJURED HUMAN TROPHOBLAST CELLS AND INHIBITING APOPTOSIS THEREIN

(71) Applicants: SONGPING LUO, (US); JIE GAO, (US); TING FENG, (US); XIUMING LIU, (US)

(72) Inventors: SONGPING LUO, GUANGZHOU (CN); JIE GAO, GUANGZHOU (CN); TING FENG, GUANGZHOU (CN); XIUMING LIU, GUANGZHOU (CN)

(21) Appl. No.: 14/497,310

(22) Filed: Sep. 25, 2014

(51) Int. Cl. A61K 36/39; A61K 36/35; C12N 5/073; A61K 36/185

(52) U.S. Cl. CPC A61K 36/39; A61K 36/185; A61K 36/35; C12N 5/0605; C12N 2500/76

(57) ABSTRACT

Composition useful in treating and/or preventing spontaneous abortions derived from a traditional recipe of herbal medicine. The composition contains active components from three herbal medicines: total flavonoids of Cuscuta Chinensis, total flavonoids of Taxillus Sutchuenensis, total polysaccharides of Taxillus Sutchuenensis, and total saponins of Radix Dipsaci Asperoidis. This composition shows potent effects on reducing apoptosis induced by mifepristone in trophoblast cells, likely by inhibiting the expression of Bax, Caspase-9 and Caspase-3. Its effect is more significant than progesterone, one of the compounds currently used for preventing spontaneous abortions.

### FIELD OF THE INVENTION

The present invention relates to a medical field of preventing or reducing spontaneous abortion, particular to a composition which has the effect of nourishing injured human trophoblast cells and inhibiting apoptosis therein, a cause for spontaneous abortion.

### BACKGROUND OF THE INVENTION

A miscarriage or spontaneous abortion is an untended loss of a fetus before the 20th week of pregnancy, which is a naturally occurring event. Around half of all fertilized eggs die and are lost spontaneously, usually before the woman knows she is pregnant. The miscarriage rate among known pregnancies is about 15-20%, most which occur during the first 7 weeks of pregnancy. The risk of miscarriage is higher in elder women (particularly at an age older than 40) or had previous miscarriages.

Trophoblast cells are cells forming the outer layer of a blastocyst, which provide nutrients to the embryo and develop into a large part of the placenta. Placenta provides maintenance, nourishment, and protection of the fetus during development and is vital to the survival of the fetus. Trophoblast cells are part of mother-fetal interface in direct contact with the embryo and the mother. In the micro-environment of the mother-fetal interface, cellular and molecular interactions can regulate trophoblast cell proliferation, apoptosis, plantation, endometrium and decidua, which in turn play important roles in maintaining a pregnancy. Thus, the trophoblast cell has been used as a research model to understand the causes of spontaneous abortion and a target for drug intervention in the effort to prevent spontaneous abortion.

Studies has found that the occurrence of spontaneous abortion associated with increased apoptosis. For example, Kaponis reported this phenomenon in “Coelomic cells show apoptosis via Fas/FasL system: a comparative study between healthy human pregnancies and missed miscarriages” (Human Reproduction Vol. 23, No. 5 pp. 1159-1169, 2008) and Wei reported it in “Apoptosis and p53 expression in the placental villi of females with unexplained recurrent spontaneous abortion” (Experimental And Therapeutic Medicine 7: 191-194, 2014). Such association was further indicated in U.S. Pat. No. 8,338,373, which found that an hG-CSF analogs are able to inhibit apoptosis in trophoblast cells and reduce spontaneous abortion.

Herbal medicines are known to be low in toxicity and some traditional recipes are known to have effects in reducing spontaneous abortion and used for such purpose for many years. However, they are used as raw extracts and have a complicated and uncertain composition and therefore are difficult for administration and quality control.

### SUMMARY OF THE INVENTION

Accordingly, one object of the present invention is to provide a composition comprising active components derived from herbal medicines, which are effective in promote proliferation and inhibit apoptosis in trophoblast cells and useful in reducing spontaneous abortions.

This abject is realized by isolating and identifying active components from a number of herbal medicinal materials, which are part of the traditional herbal recipe “Shou Tai Wuan” (STW hereinafter) used for reducing spontaneous abortions. The STW recipe consists of the following four ingredients: taxillus sutchuenensis (or “Sang Ji Sheng” in Chinese), cuscuta chinensis (or “Tu Si Zi”), radix dipsaci asperoidis (or “Xu Duan”), and donkey hide gelatin (or “A Jiao”). In the present invention, the active components are isolated from three of four STW's traditional ingredients (i.e., taxillus sutchuenensis, cuscuta chinensis and radix dipsaci asperoidis) without using anything from donkey hide gelatin. For the present invention, five active components were prepared from three herbal raw materials. They are flavonoids of cuscuta chinensis (refereed to as “X1”), flavonoids of taxillus sutchuenensis (“X2”), polysaccharides of taxillus sutchuenensis (“X3”), saponins of radix dipsaci asperoidis (“X4”) and alkaloids of radix dipsaci asperoidis (“X5”). In present invention, X5 was found not be necessary. Preferably, the composition of the present invention comprises an active component and excipients, and the active component consists of the following components (relative amount by weight): X1 (0.9), X2(0.007), X3(0.6), X4(0.05). More preferably, the active components consists of X1 (0.9), X2(0.00342), X3(0.09037) and X4(0.0795).

Another object of the present invention is to provide a method for nourishing human trophoblast cells with elevated apoptosis, which is likely to lead to spontaneous abortion. This object is achieved by contacting the trophoblast cells with an effective amount of a composition comprising a carrier and an active component consisting of X1, X2, X3 and X4. Preferably, the weight ratios between X1, X2, X3 and X4 are 0.9:0.007:0.6:0.05. More preferably, the ratios are 0.9:0.00342:0.09037:0.0795.

Still another object of the present invention is to provide a method for treating and/or preventing spontaneous abortion. This object is achieved by administering to a patient in need of such treatment an effective amount of a composition comprising a carrier and an active component consisting of X1, X2, X3 and X4. Preferably, the weight ratios between the X1, X2, X3 and X4 are 0.9:0.007:0.6:0.05. More preferably, the ratios are 0.9:0.00342:0.09037:0.0795.

It is understood that a person with ordinary skill in the art, based on the teaching of this invention, can easily vary the relative amounts of the components to a reasonable degree and still obtain satisfactory results. Those variations can be guided by simple tests as disclosed in the present invention to ensure maintaining the intended effects while varying the relative amount.

The various features of novelty which characterize the invention are pointed out with particularity in the claims annexed to and forming a part of this disclosure. For a better understanding of the invention, its operating advantages, and specific objects attained by its use, reference should be made to the drawings and the following description in which there are illustrated and described preferred embodiments of the invention.

### BRIEF DESCRIPTION OF THE DRAWINGS

FIG. 1 depicts the overall scheme according to which the present invention was carried out.

FIG. 2 shows the growth curve of the trophoblast cell.

FIG. 3 shows the effect on the level of mRNAs related to apoptosis in trophoblast cells treated by mifepristone for 24 hours.

### DETAILED DESCRIPTION OF THE INVENTION

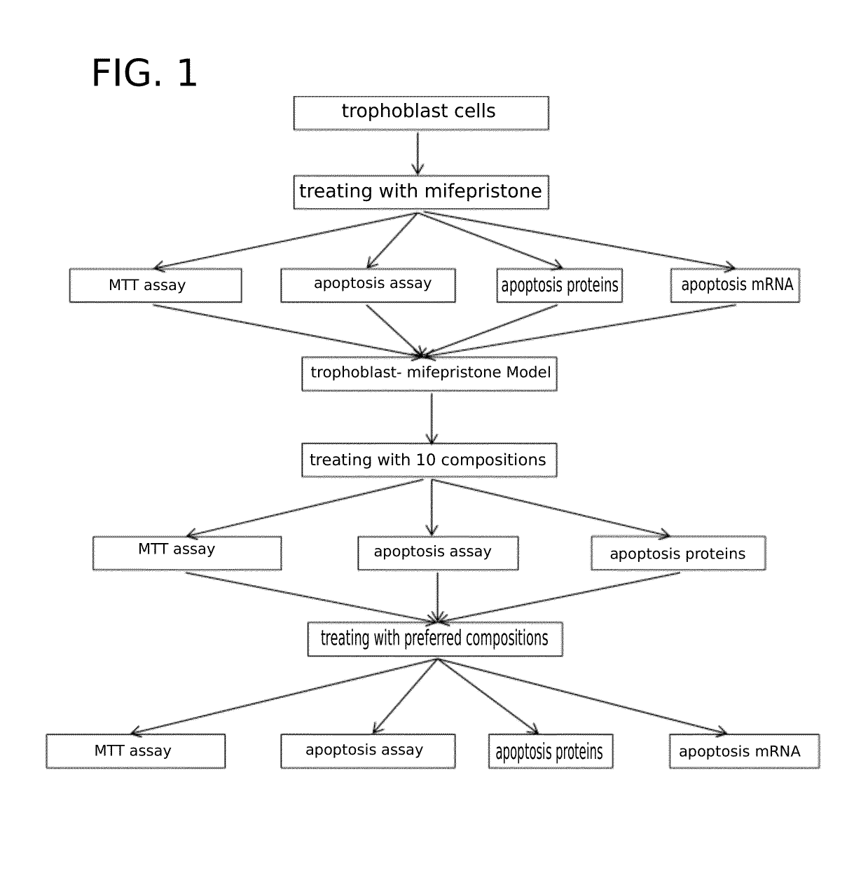

FIG. 1 presents the overall research scheme of the present invention, in which the first step is to establish a trophoblast cell model for spontaneous abortion using mifepristone, i.e., the trophoblast-mifepristone model. The model is confirmed by four assays: MTT assay, apoptosis assay, apoptosis protein detection and apoptosis mRNA detection. Once the model was established, 10 compositions was selected and tested on the model for their biological effects on cell proliferation (by MTT assay), apoptosis (by flow cytometry), apoptosis related proteins (by Western-blotting). The assay data was then subject to stepwise regression analysis, canonical correlation analysis and result optimization analysis. From the result of these analyses, a preferred composition is determined and tested in parallel with other composition and individual compounds on the same model to confirm its beneficial biological effects on the trophoblast cells.

### 1. Isolation and Culture of Trophoblast Cell

Material: with the patients' consent, chorionic villi specimens were collected from healthy women of 22-23 years of age, requesting for termination of normal pregnancy after gestation of 6-7 weeks, at the gynecologic clinic of Guangzhou University of traditional Chinese Medicine. The chorionic villus tissue was taken during the abortion procedure, quickly rinsed with saline to remove the blood, placed in the D-hanks solution within a sterile centrifuge tube, and immediately sent to the laboratory for culturing.

Cell separation: the chorionic villus tissue placed in a Petri dish, thoroughly washed with the D-hanks solution 3 times to remove residual blood. Then, the tissue was cut into pieces to a size around 1 mm, and put in a 50 ml centrifuge tube. After centrifuge (1000 rpm×5 min), discard the supernatant. The issue was added with 0.25% trypsin twice the volume of the tissue, and placed in an incubator for digestion at 37° C. for about 30 minutes. Terminate the digestion with DMEM/F12 medium containing 10% FBS. Filter the digested issue with a 200 mesh stainless steel screen, transfer the filtrate to a centrifuge tube, centrifuge (800 rpm×5 min), discard the supernatant, and resuspend the precipitate in 2 ml serum free DMEM/F12 medium.

Cell purification: Using the Percoll density gradient method with two density gradients, 35% and 45%. Add 3 ml of each density gradient into a 15 ml centrifuge tube (the higher density first) and then slowly add the cell suspension on the top of the Percoll gradients and centrifugal at 20° C., 1500 rpm/min, for 30 min. Take the cloudy middle layer of the cells and place them in another centrifuge tube. Add DMEM/F12 medium to the tube, centrifugal for 10 min at 1500 rpm, and discard the supernatant. Wait for 0.5 hour for the cells to stick to the wall of the tube and then place it in the incubator and continue the culture.

Culture conditions: the culture medium was DMEM/F12 containing 10% of fetal bovine serum, pH value of 7.0-7.2. The culture was incubated at 37° C., in 5% CO₂, with the medium changed every 48 hours and daily observation of cell growth conditions. When the cells covered 90% of the bottom surface area, digestion with 0.25% trypsin was conducted for culturing the next generation of cells.

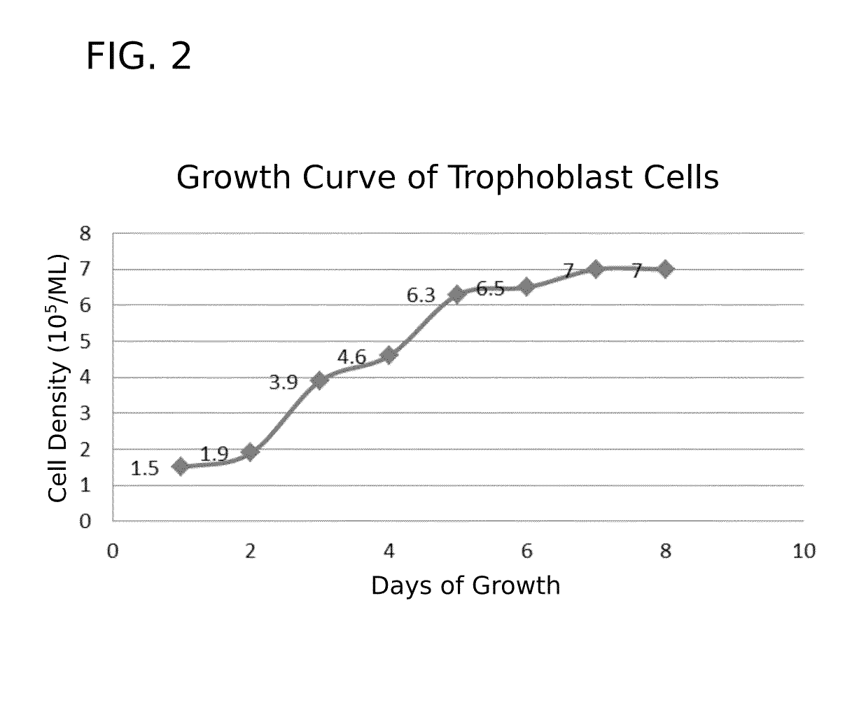

Cell growth curves: when the 4^{th} generation of cells reached a 90% coverage, the culture was digested with 0.25% trypsin and adjusted to 1×10⁵ cells/ml. Grow the cells in a 6-well plate, 1 ml per well, and count the cell number each day after the trypsin digestion. The trophoblast cell growth curve is shown in FIG. 2.

### 2. Establish of Mifepristone-Trophoblast Cell Mode for SA

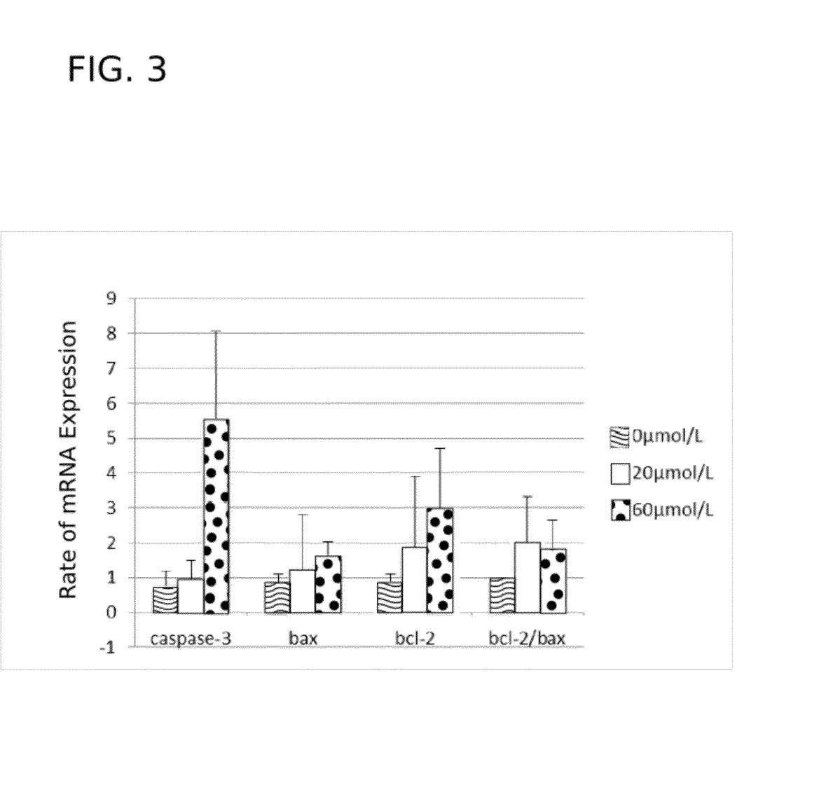

Mifepristone is an effective steroidal antagonist at the progesterone receptor. In recent years, it has been found that mifepristone can directly induce cell apoptosis. The present invention studied the effects of different concentrations of mifepristone on proliferation, apoptosis and apoptosis related protein, apoptosis gene expression in cultured trophoblastic cells (data not shown) and found that a useful model can be established at 60-100 μmol/L of mifepristone treating for 24 hours. For example, the effect of apoptotic mRNA expressions in trophoblastic cells treated by mifepristone for 24 hours is shown in FIG. 3.

### 3. Preparation of Active Herbal Components

### Flavonoids of Cuscuta Chinensis (“X1”)

Dried traditional herbal medicine cuscuta chinensis (“Tu Si Zi” in Chinese) 1000 g, bought off-the-shelf, was crushed to 20-30 mesh, soaked in 7 liter 95% ethanol for 30 minutes, subject to ultrasound at 40KHz and ˜35° C. for 30 minutes, and filtered with four layers of gauze. The filter residue was dried by vaporization at room temperature, soaked in 7 liter distilled water for 30 minutes, then boiled for 30 minutes, and filtered with four layers of gauze to obtain the first water extract. The filter residue was gain soaked in 7 liter distilled water for 30 minutes, then boiled for 30 minutes, and filtered with four layers of gauze to obtain the second water extract. The first and second water extracts were combined and condensed into 1.5 liter with a rotary evaporator vacuum at 60° C., 70 rpm and 0.1 atmospheric pressure. After cooling down, the extract was added with 1.5 liter 95% ethanol and mixed thoroughly. Put the mixture aside for 15 minutes and, when flocculent precipitate occurs, filter it with four layers of gauze. The filtrate was concentrated into a paste state with a rotary evaporator vacuum at 60° C., 70 rpm and 0.1 atmospheric pressure.

Take 16 g of the ethanol precipitated paste of cuscuta chinensis as prepared above and dissolve it in 500 ml distilled water. The resulting water solution was applied to a macroporous resin column (macroporous resin Model D101, weight 400 g). After static adsorption for 5 house, the column was in turn eluted with water, 30% ethanol and 60% ethanol (3.2 liters each). The two ethanol eluting solutions were collected, combined, and concentrated into a paste in a vacuum evaporator at 60° C., 70 rpm and 0.1 atmospheric pressure. This paste is referred to as total flavonoids of Cuscuta Chinensis or “X1” component.

### Flavonoids of Taxillus Sutchuenensis (“X2”)

1. Preparation of ethanol extract: Dried traditional herbal medicine taxillus sutchuenensis (“Sang Ji Sheng” in Chinese) 1000 g, bought off-the-shelf, was crushed crushed into 20-30 mesh, soaked in 7 liter 95% ethanol for 30 minutes, subject to ultrasound at 40 KHz and ˜35° C. for 30 minutes (in an ultrasonic extraction instrument, model THC-50B, made by Jining Tianhua ultrasonic electronic company), and filtered with four layers of gauze to obtain the first extract. The filter residue was again soaked in 7 liter 95% ethanol for 30 minutes, subject to ultrasound at 40KHz and ˜35° C. for 30 minutes, and filtered with four layers of gauze to obtain the second extract. The first and second extracts were combined and condensed into a dried paste with a rotary evaporator vacuum at 55-55° C., 70 rpm and 0.1 atmospheric pressure. This dried paste is referred to as the ethanol extract of taxillus sutchuenensis. The filter reside after the twice ethanol extractions described above was then used for preparing the water extract as described below.

2. Preparation of water extract: The filter residue after the second ethanol extraction above was dried by evaporation at room temperature to get rid of the residual ethanol. Then it was soaked in 7 liter distilled water for 30 minutes, boiled for 30 minutes, and filtered with four layers of gauze to obtain the first water extract. The filter residue was again soaked in 7 liter distilled water for 30 minutes, boiled for 30 minutes, and filtered with four layers of gauze to obtain the second water extract. The first and second water extracts were combined and condensed into a dried paste in a rotary evaporator vacuum at 60° C., 70 rpm and 0.1 atmospheric pressure. This dried paste is referred to as the water extract of taxillus sutchuenensis.

3. Preparation of X2: Take 22.2 g of the ethanol extract prepared above and dissolve it in 500 ml of distilled water. The water solution was add with 500 ml of petroleum ether in a separatory funnel and mixed by shaking. Put it aside for 30 min until it stratified, and transfer the lower layer to another separatory funnel, to which 500 ml ethyl acetate was added. After shaking the funnel for mixing, put it aside for 30 min until it was stratified and then take the lower water layer (referred to as Part A).

Take 50 g of the water extract prepared above and dissolve it 1000 ml distilled water. The water solution was applied to a macroporous resin column (weight 800 g). After static adsorption for 5 hours, the column was eluted in turn with water, 30% ethanol and 60% ethanol (each the eluting solvent was 4 liters). Each eluting solution was collected separately and concentrated to 500 ml, referred to as Part B, Part C and Part D, respectively.

Part A and Part C were combined, mixed and applied to a macroporous resin column (weight 500 g). After static adsorption for 5 hours, the column was eluted in turn with water, 10% ethanol, 30% ethanol and 60% ethanol (4 liters each) . The water elute was collected and concentrated to 500 ml, which is referred to as Part E. The 10% ethanol elute was collected and concentrated to a paste, which is referred to as 10% flavone. The 30% ethanol elute was collected and concentrated to a paste, which is referred to as 30% flavone. The 60% ethanol elute was collected, combined with Part D, and concentrated to a paste, which is referred to as 60% falvone. Combining the 10% flavone, 30% flavone and 60% flavone preparations results in the total flavonoids of taxillus sutchuenensis or the X2 component.

### Polysaccharides of Taxillus Sutchuenensis (“X3”)

From the foregoing procedure, Part B and Part E were combined and mixed.

The mixture was added with ethanol to an ethanol concentration of 75% and stored overnight for precipitation. The precipitate was collected after filtering with four layers of gauze and dried in a rotary evaporator vacuum at 60° C., 70 rpm and 0.1 atmospheric pressure to obtain the component X3 or polysaccharides of taxillus sutchuenensis.

### Saponins and Alkaloids of Radix Dipsaci Asperoidis (“X4” and “X5”)

Dried traditional herbal medicine radix dipsaci asperoidis (“Xu Duan” in Chinese) 3500 g, bought off-the-shelf, was crushed crushed into 20-30 mesh, subject to reflux extraction in six times (by weight) the amount of 75% ethanol for 2 hours, and filtered with four layers of gauze. The filtration residue was subject for a second reflux extraction in six times the amount of 75% ethanol for 1.5 hours and filtered with four layers of gauze. The filtration residue was once again subject to a third reflux extraction in six times the amount of 75% ethanol for 1.5 hours. The filtrates from three extractions were combined and concentrated to a paste under a reduced pressure in a rotary evaporator vacuum at 50-55° C., 70 rpm and 0.1 atmospheric pressure. This paste is referred to as the alcohol extract of radix dipsaci asperoidis.

The above alcohol extract was applied to a HP-20 macroporous resin column, which was then eluted with water and 95% ethanol. The elute was put in a rotary evaporator to remove ethanol, resulting a mixture containing total alkaloids and total glycosides. Thereafter, the mixture was acidified with 1% dilute hydrochloric acid to adjust the pH to 1-2 and extracted with chloroform to obtain non-polar components of total 16.1 g (i.e., an extraction rate of 0.46%). The remaining aqueous layer was neutralized with 1% NaOH solution, adjusted to a pH value 10-11, and extracted with chloroform. The chloroform extract was concentrated under a reduced pressure to obtain a total alkaloids of 5.36 g (an extraction rate of 0.153%), which is referred to as the X5 component. The remaining layer was then neutralized with 1% hydrochloric acid and concentrated under a reduced pressure to obtain a total glycosides 775 g (an extraction rate of 22.5%), which is referred to as the X4 component.

### 4. Homogeneous Design for Identifying Active Components

Ten Compositions Formulated from 5 Components:

Homogeneous design table: In this study, the test factor is 5, and based on the formula m/2+1=5, m=8 and n=8+1=9, where m is the maximum number of factors can be arranged, n is the number of tests. According to the experimental design, it requires a dose of 0 so that there are nine gradient doses plus a zero dose. Using homogeneous design table U*10 (10⁸), D=0.2414, ten compositions were formulated according to the following table.

| | X1 | X2 | X3 | X4 | X5 |
|---|---|---|---|---|---|
| Composition | (mg/ml) | (mg/ml) | (mg/ml) | (mg/ml) | (mg/ml) |
| 1 | 0 | 0.002 | 0.3 | 0.04 | 0.008 |
| 2 | 0.1 | 0.005 | 0.7 | 0.09 | 0.006 |
| 3 | 0.2 | 0.008 | 0 | 0.03 | 0.004 |
| 4 | 0.3 | 0 | 0.4 | 0.08 | 0.002 |
| 5 | 0.4 | 0.003 | 0.8 | 0.02 | 0 |
| 6 | 0.5 | 0.006 | 0.1 | 0.07 | 0.009 |
| 7 | 0.6 | 0.009 | 0.5 | 0.01 | 0.007 |
| 8 | 0.7 | 0.001 | 0.9 | 0.06 | 0.005 |
| 9 | 0.8 | 0.004 | 0.2 | 0 | 0.003 |
| 10 | 0.9 | 0.007 | 0.6 | 0.05 | 0.001 |

These 10 compositions were tested parallel on the Mifepristone-Trophoblast Cell Model by observing the effects of each composition on the following biological activities: (a) cell proliferation, (b) early state apoptosis, (b) Caspase-3 expression, (c) HCG secretion.

The experimental data on the cell proliferation rate, early apoptosis rate, Caspase-3 protein expression and cell supernatants HCG level expression were then subject to stepwise regression analysis, canonical correlation analysis and result optimization analysis. From these analyses, it is concluded that the preferred ratio among X1:X2:X3:X4:X5 is 0.9:0.00342:0.09037:0.0795:0. For the sake of brevity, the details of the experimental data are not fully shown here, but are exemplified by the data in the following table, which shows the effects of each of the 10 compositions on early stage apoptosis on the Mifepristone-Trophoblast Cell Model, where “Blank” refers to normal trophoblast cells not being treated with anything but given only the DMEM/F12 medium as control, “Model” means the Mifepristone-Trophoblast Cell Model established with mifepristone (60 μl/ml in DMEM/F12, 24 h) and given only the DMEM/F12 medium, and “Comp 1” means the same model cells being treated with Composition 1 in the DMEM/F12 medium. The number means the percentage of the cells underwent apoptosis.

Effects on Early Stage Apoptosis of Various Herbal Components

| | |
|---|---|
| Num- | |
| ber of | Duration of treatment |

| Group | tests | 24 h (%) | 48 h (%) |
|---|---|---|---|
| Blank | 3 | 5.033 ± 0.115 | 4.933 ± 0.208 |
| Model | 3 | 25.967 ± 2.754* | 18.267 ± 0.503** |
| Comp 1 | 3 | 22.267 ± 8.450 | 22.933 ± 8.193 |
| Comp 2 | 3 | 18.767 ± 6.966 | 20.333 ± 5.707 |
| Comp 3 | 3 | 19.200 ± 6.458 | 21.767 ± 1.721* |
| Comp 4 | 3 | 13.700 ± 1.967 | 14.533 ± 6.525 |
| Comp 5 | 3 | 19.967 ± 5.493 | 17.500 ± 5.122 |
| Comp 6 | 3 | 18.733 ± 3.564 | 21.233 ± 3.002 |
| Comp 7 | 3 | 22.100 ± 9.338 | 22.533 ± 5.947 |
| Comp 8 | 3 | 18.933 ± 6.615 | 14.233 ± 6.208 |
| Comp 9 | 3 | 15.367 ± 5.472 | 12.633 ± 2.702 |
| Comp 10 | 3 | 14.700 ± 1.908 | 12.267 ± 2.324 |

### 5. Comparative Study on Preferred Composition

### Grouping and Administration:

The testing is divided into eight groups as follows:

(1) Normal group: normal cells, given DMEM/F12 medium; (2) Model group: model cells (established by culturing for 24 hours in DMEM/F12 medium containing 60 μmol/L mifepristone and discarding the supernatant), given DMEM/F12 medium; (3) Positive control group: model cells, given 0.02 mg/ml progesterone in DMEM/F12 medium; (4) Preferred composition group: model cells, given a solution containing X1 (0.9 mg/ml), X2 (0.00342 mg/ml), X3 (0.09037 mg/ml), and X4 (0.0795 mg/ml) in DMEM/F12 medium; (5) X1 group: model cells, given X1 (1 mg/ml) in DMEM/F12 medium; (6) Optimized composition 10 group: model cells, given a solution containing X1(0.9 mg/ml), X2 (0.07 mg/ml), X3 (0.6 mg/ml), and X4 (0.05 mg/ml) in DMEM/F12 medium; (7) Hyperoside group: model cells, given hyperoside (10⁻⁴ mole/L) in DMEM/F12 medium; and (8) Asperosaponin group: model cells, given asperosaponin (10⁻⁴ mol/L) in DMEM/F12 medium. Hyperoside and asperosaponin are individual compounds isolated from herbal medicines cuscuta chinensis and taxillus sutchuenensis, respectively. Progesterone is a compound often used to prevent spontaneous abortion and was used in present invention as positive treatment control for comparison with of the novel composition of the present invention.

### MTT Assay on Cell Proliferation:

The trophoblast cells (adjust to 1.5×10⁴/ml) were seeded in wells on a 96-well plate, 1 ml per well. After incubating for 48 hours at 37° C., 5% CO₂, the cells were grouped and treated accordingly (see above). Assays were conducted after treatment for 24 h and 48 h according to the following procedure:

Add 10 μl 1×MTT (3-(4,5-dimethylthiazol-2-yl)-2,5-diphenyltetrazolium bromide) to the well and continue incubation at 37° C., 5% CO2 for 4 hours to reduce MTT to formazan. Remove the supernatant from the well and add to each well 150 μl DMSO (dimethylsulfoxide) to dissolve formazan. After shaking the 96 plate for 10 min to make crystals completely dissolved, determine the OD value at 490 nm with a enzyme immunoassay analyzer. The determination was repeated three times.

### Radioimmunoassay on HCG Expression in Cell Supernatant:

The cells (4×10⁵ cells/ml) were seeded in the 6-well plate, 1 ml per well, and incubated at 37° C., 5% CO₂ for 48 hours. Then, the cells were grouped and treated accordingly (see above). After 48 hour treatment, cell supernatants were collected and sent to the Department of Nuclear Medicine, Guangzhou University of Traditional Chinese Medicine, for measuring HCG (human chorionic gonadotropin) via a routine radioimmunoassay procedure.

### Annexin V/PI Assay on Cell Apoptosis:

The cells 4×10⁵ cells/ml were seeded in the 6-well plate, 1 ml per well and incubated at 37° C., 5% CO₂ for 48 h. Then, the cells were grouped and treated accordingly (see above). The assays were conducted as follows:

At the end of the predetermined treatment, the cells were digested and spun down(2000 rpm×5 min centrifugation). Discard the medium, wash the cells twice with cold PBS (resuspension and then 2000 rpm×5 min centrifugation). Remove the supernatant and resuspend the cell in 400 μl 1×Annexin V binding solution. Then, add 5 μl AnnexinV-EGFP staining solution to the cell suspension and mix gently. Incubate the cells at 2-8° C. in dark for 15 minutes. Add 10 μl PI (propidium iodide) and mix gently and incubate the cells in dark at 2-8° C. for 5 min. Detection by flow cytometry was conducted within one hour. The detection was repeated three times.

Western Blot Assay on Caspase-3, bax, bcl-2,Caspase-9 and XIA:

Extraction of total cellular protein and cytoplasmic protein: The cells 4×10⁵ cells/ml were seeded in the 6-well plate. After incubating at 37° C., 5% CO₂ for 44 hours, the cells are grouped and treated according to the experiment scheme (see above). Extraction conducted at two treatment points: 24-24 h (treat with agent for 24 hours and continue incubation for 24 hours after removing the agent) and 24-48 h (treat with agent for 24 hours and continue incubation for 48 hours after removing the agent). The assays were conducted as follows:

At each predetermined time point, discard the culture medium, wash the cells three times with cold PBS solution, 3 min each time. After removing PBS, put the cells on ice. Add lysis buffer, 150 μl per well, and then place the cells on ice for 5 min, scrape the surface cells and transfer the cell lysate to an EP tube. Put the tube on ice for 30 min. After centrifuge (4° C., 12000 rpm/min, 15 min), transfer the supernatant to the EP tube and store it at −20° C. Determine the protein concentration with the BCA method and adjust the protein concentration to the same level. Add 3 μl 5× SDS-PAGE sampling buffer, denature at 100° C. for 5 min, and store the samples at −20° C. for Western Blotting. The Western Blotting procedure was conducted in a standard manner known in the art, description of which is omitted herewith for the sake of brevity.

PCR Assay on mRNAs:

Design of primers: Based on search for the sequence of the target gene mRNA in GenBank, the following primers were designed:

1. for H-BAX (81 bP)
Forward Primer:
SEQ ID NO: 1
5′-CAT GTT TTC TGA CGG CAA CTT C-3′
Reverse Primer:
SEQ ID NO: 2
5′-AGG GCC TTG AGC ACC AGT TT-3′
Probe:
SEQ ID NO: 3
5′-FAM-TGT CGC CCT TTT CTA CTT TGC-TAMRA-3′
2. for H-BCL-2 (67 bP)
Forward Primer:
SEQ ID NO: 4
5′-TGG GAT GCC TTT GTG GAA CT-3′
Reverse Primer:
SEQ ID NO: 5
5′-GAG ACA GCC AGG AGA AAT CAA AC-3′
Probe:
SEQ ID NO: 6
5′-FAM-TAT GGC CCC AGC ATG CGA CCT C-TAMRA-3′
3. for H-CASP3 (100 bP)
Forward Primer:
SEQ ID NO: 7
5′-TACCAGTGGAGGCCGACTTC-3′
Reverse Primer:
SEQ ID NO: 8
5′-CAAAGCGACTGGATGAACCA-3′
Probe:
SEQ ID NO: 9
5′-FAM-TCCACAGCACCTGGTTATTATTCTTGGCG-TAMRA-3′
4. for H-GAPDH (111 bP)
Forward Primer:
SEQ ID NO: 10
5′-CCTGCACCACCAACTGCTTAG-3′
Reverse Primer:
SEQ ID NO: 11
5′-CAGTCTTCTGGGTGGCAGTGA-3′
Probe:
SEQ ID NO: 12
5′-FAM-CATCCATGACAACTTTGGTATCGTG-TAMRA-3′
5. for XIAP (111 bP)
Forward Primer:
SEQ ID NO: 13
5′-AGTGCTTTTGTTGTGGTGGAAA-3′
Reverse Primer:
SEQ ID NO: 14
5′-GGCCCAAAACAAAGAAGCAA-3′
Probe:
SEQ ID NO: 15
5′-CCTGGTCAGAACACAGGCGACACTTTC-3′
6. for CasPase-9 (111 bP)
Forward Primer:
SEQ ID NO: 16
5′-AACAGGCAAGCAGCAAAGTTG-3′
Reverse Primer:
SEQ ID NO: 17
5′-GGGTGTTTCCGGTCTGAGAA-3′
Probe:
SEQ ID NO: 18
5′-CCCCAGTGGTGCTCAGACCAGAGATTC-3′

Total RNA extraction: Cells 4×10⁵ cells/ml were seeded in the 6-well plate, 1 ml per well, and incubated at 37° C., 5% CO₂ for 48 h. Then, the cells were grouped and treated according to the experiment scheme (see above). Extraction was conducted at two time points: 24 h and 24-24 h (24 hours of agent treatment and 24 hours after removing the agent), according to the procedures described as follows:

Placed the cells in the 1.5 ml EP tube, add Trizol 1 ml, and mix by shaking. Add chloroform 0.2 ml, cover tightly, and shake vigorously for 15 s. Store it at 4° C. for 2-3 min, centrifugal at 4° C., 12000 rpm×15 min. Transfer the supernatant to a new 1.5 ml EP tube, add an equal volume of isopropanol, incubate at 4° C. for 10 min, and centrifuge 4° C., 12000 rpm×10 min. After discarding the supernatant, add 75% ethanol (containing DEPC water) to wash the precipitate once, centrifugal 4° C. 7500 rpm×5 min, and discard ethanol. Air or vacuum dry for 5-10 min (not completely dry), add DEPC water to dissolve RNA, store at −80° C. for further process.

Reverse transcription: In a sterile 0.2 ml centrifuge tubes, prepare the reaction according to the following:

| | | | |
|---|---|---|---|
| Oligo(dt) | 1 | ul | |
| DEPC water | 6 | ul→ | 70° C. for 5 min, then place on ice → |
| RNA | 5 | ul | |
| 5 × Reaction | 4 | ul | |
| Buffer(Buffer- | | | |
| m-Mulvrt) | | | |
| Ribolock | 1 | ul → | 37° C. for 5 min → |
| R10mmdNTPmix | 2 | ul | |
| m-muLvRT | 1 | ul → | 42° C. for 60 min, 70° C. for 10 min, |
| | | | store at −20° C. |

Quantitative PCR:experimental design is in accordance with standard ΔΔCt analytical method. The experimental procedure is as follows:

(1) The 2×AllinOne™ Q-PCR Mix thawed at room temperature, gently mix and centrifuge briefly.

(2) Prepare reaction mix (performed on ice):

| Agent | Amount | End concentration |
|---|---|---|
| 2 × AllinOneTM Q-PCR Mix | 10 ul | 1× |
| ddH2O | 2 ul | |
| PCR Forward Primer(2 μM) | 2 ul | 0.2 μM |
| PCR Reverse Primer(2 μM) | 2 ul | 0.2 Mm |
| cDNA(1:5dilute) | 2 ul | 0.2 μM |
| PROBE | 1 ul | |
| Total volume | 20 ul | |

(3) PCR reaction: using a standard three-step reaction procedure:

| Number of cycles Process | | Temperature | Duration | Detection |
|---|---|---|---|---|
| 1 | Pre-denaturation | 93° C. | 2 min | off |
| | Denaturation | 93° C. | 30 sec | off |
| 40 | Extension | 55° C. | 45 sec | on |

### Statistic Treatment:

All the experimental data were processed and analyzed by SPSS 18.0 software and the results are expressed in the form of mean±standard deviation. For data meeting the requirements for normality and homogeneity of variance, multiple samples were compared using ANOVA (One-Way ANOVA) test, while variance arrhythmia is tested using Dunnett T3. P<0.05 indicates a statistically significant. The experiments were repeated three times.

### Assay Results:

### (1) On Cells Proliferation:

The following table shows the effects of various compositions on the proliferation rate of trophoblastic cells in the mifepristone induced model. The data was obtained from MTT assays. The number means the cell proliferation rate as compared to the Blank.

| | Num- | | |
|---|---|---|---|
| | ber of | Duration of Treatment | |
| Group | Tests | 24 h | 48 h |
| Blank | 3 | 1.0000 | 1.0000 |
| Model | 3 | 0.686 ± 0.025* | 0.757 ± 0.022* |
| Positive | 3 | 0.805 ± 0.030* | 0.825 ± 0.061 |
| Control | | | |
| X1 | 3 | 0.814 ± 0.096 | 0.997 ± 0.141 |
| Optimized | 3 | 0.877 ± 0.028^{Δ} | 0.970 ± 0.166 |
| Composition | | | |
| 10 | | | |
| Hyperoside | 3 | 0.788 ± 0.072 | 0.869 ± 0.029 |
| Asperosaponin | 3 | 0.787 ± 0.086 | 0.809 ± 0.019* |
| Preferred | 3 | 0.953 ± 0.010^{ΔΔ} | 1.028 ± 0.103 |
| Composition | | | |

*compared with Positive Control P < 0.05;
^{Δ}compared with Model P < 0.05;
^{ΔΔ}compared with Model P < 0.01.

### (2) On β-HCG Expression:

The following table shows the effects of various composition on the β-HCG level in trophoblastic cells in the mifepristone induced model.

| | Num- | |
|---|---|---|
| | ber of | Duration of Treatment |
| Group | Tests | 48 h (MIU/ML) |
| Blank | 3 | 1.237 ± 0.107* |
| Model | 3 | 0.923 ± 0.064* |
| Positive | 3 | 1.067 ± 0.038*^{Δ} |
| Control | | |
| X1 | 3 | 1.083 ± 0.015*^{Δ} |
| Optimized | 3 | 3.230 ± 0.076 |
| Composition | | |
| 10 | | |
| Hyperoside | 3 | 1.377 ± 0.142* |
| Asperosaponin | 3 | 0.870 ± 0.026* |
| Preferred | 3 | 5.410 ± 1.963 |
| Composition | | |

*compared with Optimized Composition 10 P < 0.05;
^{Δ}compared with Asperosaponin P < 0.05.

### 3. On Early Stage Apoptosis:

The following table shows the effects of various composition on early stage apoptosis of trophoblastic cells in the mifepristone induced model. The data was obtained from Annexin V FITC/PI assays. The number means the percentage of cells underwent apoptosis.

| | Num- | | |
|---|---|---|---|
| | ber of | Duration of Treatment | |
| Group | Tests | 24 h (%) | 48 h (%) |
| Blank | 3 | 4.900 ± 0.173 | 4.267 ± 1.021 |
| Model | 3 | 23.800 ± 2.163 ** | 21.133 ± 1.940 ** |
| Positive | 3 | 17.400 ± 1.229 **^{ΔΔ} | 16.367 ± 1.447 **^{▴} |
| Control | | | |
| X1 | 3 | 13.767 ± 1.002 **^{ΔΔ} | 12.600 ± 1.30 8*^{Δ} |
| Optimized | 3 | 14.500 ± 3.487 **^{ΔΔ} | 12.667 ± 1.159 ** |
| Composition | | | |
| 10 | | | |
| Hyperoside | 3 | 16.367 ± 1.942 **^{ΔΔ} | 15.633 ± 2.136 ** |
| Asperosaponin | 3 | 16.067 ± 2.974 **^{ΔΔ} | 15.933 ± 3.742 ** |
| Preferred | 3 | 11.300 ± 4.158 **^{ΔΔ} | 9.833 ± 1.172 *^{Δ} |
| Composition | | | |

* compared Blank P < 0.05;
** compared with Positive Control P < 0.01;
^{Δ}compared with Model P < 0.05,
^{ΔΔ}compared with Model P < 0.01;
^{▴}compared with Preferred Composition P < 0.05.

### 4. On Caspase-3 Expression:

The following table shows the effects of various composition on Caspase-3 expression in trophoblastic cells in the mifepristone induced model. The data was obtained from Western-Blot assays, and the number means the relative amount of protein compared with Blank.

| | Num- | | |
|---|---|---|---|
| | ber of | Duration of Treatment | |
| Group | Tests | 24 h | 48 h |
| Blank | 3 | 1.000± | 1.000 |
| Model | 3 | 3.527 ± 0.485 | 2.137 ± 0.266** |
| Positive | 3 | 2.336 ± 0.483 | 2.158 ± 0.298** |
| Control | | | |
| X1 | 3 | 1.860 ± 0.192 | 1.72 ± 0.239**^{▴} |
| Optimized | 3 | 1.190 ± 0.113 | 1.181 ± 0.378^{ΔΔ▴▴▾} |
| Composition | | | |
| 10 | | | |
| Hyperoside | 3 | 1.847 ± 0.123* | 1.648 ± 0.206**^{Δ▴} |
| Asperosaponin | 3 | 1.372 ± 0.338^{Δ} | 1.508 ± 0.217*^{ΔΔ▴▴□} |
| Preferred | 3 | 1.091 ± 0.050 | 0.999 ± 0.219^{ΔΔ▴▴▾} |
| Composition | | | |

*compared Blank P < 0.05;
**compared with Blank P < 0.01;
with the control group;
^{Δ}compared with Model P < 0.05,;
^{ΔΔ}compared with Model P < 0.01;
^{▴}compared with Positive Control P < 0.05;
^{▴▴}compared with Positive Control P < 0.01;
^{▾}compared with X1 P < 0.05;
^{□}compared with Preferable Composition P < 0.05.

### 5. On Caspase-9 Expression:

The following table shows the effects of various composition on Caspase-9 expression in trophoblastic cells in the mifepristone induced model. The data was obtained from Western-Blot assays, and the number means the relative amount of protein compared with Blank.

| | |
|---|---|
| Num- | |
| ber of | Duration of Treatment |

| Group | Tests | 24 h | 48 h |
|---|---|---|---|
| Blank | 3 | 1.000 | 1.000 |
| Model | 3 | 3.781 ± 0.359* | 2.723 ± 0.323 |
| Positive | 3 | 3.270 ± 0.602 | 2.477 ± 0.355 |
| Control | | | |
| X1 | 3 | 2.605 ± 1.073 | 2.286 ± 0.348 |
| Optimized | 3 | 1.491 ± 0.373^{Δ} | 1.578 ± 0.332 |
| Composition | | | |
| 10 | | | |
| Hyperoside | 3 | 2.079 ± 0.267^{Δ} | 2.221 ± 0.651 |
| Asperosaponin | 3 | 1.962 ± 0.273^{Δ} | 1.887 ± 0.742 |
| Preferred | 3 | 1.125 ± 0.173^{Δ} | 1.295 ± 0.310 |
| Composition | | | |

*compared with Blank P < 0.05;
^{Δ}compared with Model P < 0.05.

### 5. On Bax Expression:

The following table shows the effects of various composition on Bax expression in trophoblastic cells in the mifepristone induced model. The data was obtained from Western-Blot assays, and the number means the relative amount of protein compared with Blank.

| | Num- | | |
|---|---|---|---|
| | ber of | Duration of Treatment | |
| Group | Tests | 24 h | 48 h |
| Blank | 3 | 1.000 | 1.000^{Δ} |
| Model | 3 | 3.487 ± 0.507 ** | 3.159 ± 0.218 |
| Positive | 3 | 2.964 ± 0.395 ** | 2.135 ± 0.591 |
| Control | | | |
| X1 | 3 | 2.494 ± 0.279 **^{ΔΔ▴▴} | 2.070 ± 0.804 |
| Optimized | 3 | 1.197 ± 0.051^{ΔΔ▴▴▾▾} | 1.496 ± 0.505 |
| Composition | | | |
| 10 | | | |
| Hyperoside | 3 | 1.687 ± 0.519*^{ΔΔ▴▴▾▾} | 2.544 ± 0.383 |
| Asperosaponin | 3 | 1.503 ± 0.180^{ΔΔ▾▾} | 1.804 ± 0.558 |
| Preferred | 3 | 1.197 ± 0.189^{ΔΔ▴▴▾▾} | 1.442 ± 0.383^{Δ} |
| Composition | | | |

*compared with Blank P < 0.05;
** compared with Blank [symbol] P < 0.01;
^{Δ}compared with Model P < 0.05;
^{ΔΔ}compared with Model P < 0.01;
^{▴▴}compared with Positive Control P < 0.01;
^{▾▾}compared with X1 P < 0.05.

### 5. On BaI-2/Bax Expression Ratio:

The following table shows the effects of various composition on BcI-2/Bax expression ratio in trophoblastic cells in the mifepristone induced model. The data was obtained from Western-Blot assays, and the number means the relative ratio of the proteins compared with Blank.

| | Num- | | |
|---|---|---|---|
| | ber of | Duration of Treatment | |
| Group | Tests | 24 h | 48 h |
| Blank | 3 | 1.000 | 1.000 |
| Model | 3 | 0.358 ± 0.100 | 0.341 ± 0.062* |
| Positive | 3 | 0.537 ± 0.051 | 0.900 ± 0.404 |
| Control | | | |
| X1 | 3 | 0.884 ± 0.344 | 0.520 ± 0.058 |
| Optimized | 3 | 1.586 ± 0.476^{ΔΔ∇∇▴} | 1.401 ± 0.460 |
| Composition | | | |
| 10 | | | |
| Hyperoside | 3 | 1.544 ± 0.726^{ΔΔ∇∇} | 0.518 ± 0.186 |
| Asperosaponin | 3 | 1.905 ± 0.297**^{ΔΔ∇∇▴▴} | 1.503 ± 0.454 |
| Preferred | 3 | 2.287 ± 0.577**^{ΔΔ∇∇▴▴▾} | 1.884 ± 0.314 |
| Composition | | | |

*compared with Blank P < 0.05;
**compared with Blank P < 0.01;
^{ΔΔ}compared with Model P < 0.01;
^{∇∇}compared with Positive Control P < 0.01;
^{▴}compared with X1 P < 0.05;
^{▴▴}compared with X1 P < 0.01;
^{▾}compared with Optimized Composition 10 P < 0.05.

### 6. On XIAP Expression:

The following table shows the effects of various compositions on XIAP expression in trophoblastic cells in the mifepristone induced model. The data was obtained from Western-Blot assays, and the number means the relative amount of protein compared with Blank.

| | |
|---|---|
| Num- | |
| ber of | Duration of Treatment |

| Group | Tests | 24 h | 48 h |
|---|---|---|---|
| Blank | 3 | 1.000 | 1.000 |
| Model | 3 | 0.969 ± 0.103 | 1.421 ± 0.387 |
| Positive | 3 | 1.761 ± 0.415 | 1.576 ± 0.322 |
| Control | | | |
| X1 | 3 | 1.231 ± 0.656 | 0.868 ± 0.385 |
| Optimized | 3 | 2.292 ± 0.784 | 1.857 ± 0.661 |
| Composition | | | |
| 10 | | | |
| Hyperoside | 3 | 1.528 ± 0.317 | 1.673 ± 0.768 |
| Asperosaponin | 3 | 2.127 ± 0.421 | 1.706 ± 0.492 |
| Preferred | 3 | 2.422 ± 0.726 | 2.622 ± 1.549 |
| Composition | | | |

7. On mRNAs Related to Apoptosis:

The following table shows the effects of various compositions on levels of mRNAs related to apoptosis in trophoblastic cells in the mifepristone induced model.

| | | | | | Bcl-2 mRNA/ | Caspase- | Caspase- |
|---|---|---|---|---|---|---|---|
| Group | No | XIAPmRNA | BaxmRNA | Bcl-2 mRNA | BaxmRNA | 3 mRNA | 9 mRNA |
| Blank | 3 | 0.872 ± 0.222 | 1.936 ± 1.622^{ΔΔ} | 0.998 ± 0.003 | 0.754 ± 0.426 | 1.697 ± 1.207^{ΔΔ} | 1.128 ± 0.221 |
| Model | 3 | 0.501 ± 0.326 | 7.954 ± 0.584 | 1.271 ± 1.501 | 0.169 ± 0.207 | 7.353 ± 2.153 | 3.576 ± 1.283 |
| Positive | 3 | 1.037 ± 0.425 | 0.442 ± 0.442^{ΔΔ} | 2.971 ± 2.839 | 7.351 ± 6.001 | 3.089 ± 1.778^{ΔΔ} | 1.286 ± 0.780 |
| Control | 3 | 1.222 ± 0.302 | 0.510 ± 0.510^{ΔΔ} | 2.918 ± 3.123 | 4.868 ± 3.99 | 3.295 ± 0.334^{ΔΔ} | 2.324 ± 2.051 |
| X1 | | | | | | | |
| Optimized | 3 | 1.650 ± 1.122 | 0.318 ± 0.151^{ΔΔ} | 4.236 ± 2.646 | 12.538 ± 3.133 | 3.471 ± 0.566 | 1.385 ± 0.913 |
| composition | | | | | | | |
| 10 | 3 | 1.315 ± 0.706 | 2.469 ± 1.058^{Δ} | 2.726 ± 1.419 | 1.470 ± 1.321 | 4.640 ± 1.865^{Δ} | 3.512 ± 3.828 |
| Hyperoside | | | | | | | |
| Asperosaponin | 3 | 1.253 ± 1.540 | 3.181 ± 1.576 | 4.908 ± 3.037 | 1.88 ± 1.196 | 2.783 ± 0.541 | 1.240 ± 0.945 |
| Preferred | 3 | 1.526 ± 0.536 | 1.401 ± 0.357^{ΔΔ} | 3.587 ± 2.606 | 2.682 ± 1.875 | 3.372 ± 0.108^{ΔΔ} | 1.198 ± 0.057 |
| Composition | | | | | | | |

^{Δ}compared with Model P < 0.05;
^{ΔΔ}compared with Model P < 0.01.

The foregoing results demonstrate that the composition of present invention can overcome the apoptosis induced by mifepristone in trophoblast cells. While not wishing to be constrained by theory, such effect on apoptosis is likely to be achieved by inhibiting the expression of Bax, Caspase-9 and Caspase-3. The results also demonstrate that while the individual compounds of the herbal extract showed effects on inhibiting apoptosis in trophoblast cells, they are less potent compared with the optimized composition 10 and the preferred composition of the present invention. Finally, the results suggested that not all ingredients that are traditionally believed to be important for treatment of spontaneous abortion are necessary in the herbal recipe. Thus, according to the present invention, the preferred composition is much simpled in terms of type of ingredients necessary for realizing the intended effects, compared to the traditional recipe and is easier for administration and for quality control.

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims.

What is claimed is:

1. A composition for reducing apoptosis in human trophoblast cells, comprising:
(a) a total flavonoid component of cuscuta chinensis,
(b) a total flavonoid component of taxillus sutchuenensis,
(c) a total polysaccharide component of taxillus sutchuenensis, and
(d) a total saponin component of radix dipsaci asperoidis.

2. The composition according to claim 1, where relative amounts of said components are:
(a) said flavonoid component of cuscuta chinensis: 0.9 mg,
(b) said flavonoid component of taxillus sutchuenensis: 0.007 mg,
(c) said polysaccharide component of taxillus sutchuenensis: 0.6 mg,
(d) said saponin component of radix dipsaci asperoidis: 0.05 mg.

3. The composition according to claim 1, wherein relative amounts of said components are:
(a) said flavonoid component of cuscuta chinensis: 0.9 mg,
(b) said flavonoid component of taxillus sutchuenensis: 0.00342 mg,
(c) said polysaccharide component of taxillus sutchuenensis: 0.09037 mg,
(d) said saponin component of radix dipsaci asperoidis: 0.0795 mg.

4. A method of reducing apoptosis reducing apoptosis in human trophoblast cells by contacting said cells with an effective amount of a composition comprising:
(a) a total flavonoid component of cuscuta chinensis,
(b) a total flavonoid component of taxillus sutchuenensis,
(c) a total polysaccharide component of taxillus sutchuenensis, and
(d) a total saponin component of radix dipsaci asperoidis.

5. The method according claim 4, wherein relative amounts of said components in said composition are:
(a) said flavonoid component of cuscuta chinensis: 0.9 mg,
(b) said flavonoid component of taxillus sutchuenensis: 0.007 mg,
(c) said polysaccharide component of taxillus sutchuenensis: 0.6 mg,
(d) said saponin component of radix dipsaci asperoidis: 0.05 mg.

6. The method according claim 4, wherein relative amounts of said components in said composition are:
(a) said flavonoid component of cuscuta chinensis: 0.9 mg,
(b) said flavonoid component of taxillus sutchuenensis: 0.00342 mg,
(c) said polysaccharide component of taxillus sutchuenensis: 0.09037 mg,
(d) said saponin component of radix dipsaci asperoidis: 0.0795 mg.

7. A method of preventing spontaneous abortion of a woman by adminstering an effect amount of a composition comprising:
(a) a total flavonoid component of cuscuta chinensis,
(b) a total flavonoid component of taxillus sutchuenensis,
(c) a total polysaccharide component of taxillus sutchuenensis, and
(d) a total saponin component of radix dipsaci asperoidis.

8. The method according claim 7, wherein relative amounts of said components in said composition are:
(a) said flavonoid component of cuscuta chinensis: 0.9 mg,
(b) said flavonoid component of taxillus sutchuenensis: 0.007 mg,
(c) said polysaccharide component of taxillus sutchuenensis: 0.6 mg,
(d) said saponin component of radix dipsaci asperoidis: 0.05 mg.

9. The method according claim 7, wherein relative amounts of said components in said composition are:
(a) said flavonoid component of cuscuta chinensis: 0.9 mg,
(b) said flavonoid component of taxillus sutchuenensis: 0.00342 mg,
(c) said polysaccharide component of taxillus sutchuenensis: 0.09037 mg,
(d) said saponin component of radix dipsaci asperoidis: 0.0795 mg.
